(19) United States
(12) Patent Application Publication
(10) Pub. No.: US 2025/0229053 A1
(43) Pub. Date: Jul. 17, 2025

(54) BREATHING COACH APPLICATION ON SMART WATCH

(71) Applicant: Kashtin Garret Sean Cook, Grand Prairie (CA)

(72) Inventors: Kashtin Garret Sean Cook, Grand Prairie (CA); Anthony Yuet Keung CHAN, Richmond (CA)

(21) Appl. No.: 19/068,051

(22) Filed: Mar. 3, 2025

Related U.S. Application Data
(63) Continuation-in-part of application No. 17/876,744, filed on Jul. 29, 2022.
Application No. 17/876,744 is a continuation-in-part of application No. 17/334,891, filed on May 31, 2021, now abandoned.

(51) Int. Cl. A61M 21/02; A61M 21/00

(52) U.S. Cl. CPC A61M 21/02; A61M 2021/005; A61M 2021/0088; A61M 2209/088; A61M 2230/42

(57) ABSTRACT

A breathing coach system and method is disclosed comprising a biometric device capable of measuring one or more biometric measurements of a user; an application executed on a processor on a wearable device monitoring said one or more biometric measurements coupled to said biometric device and notifying the user to perform a recommended breathing pattern to effect said one or more biometric measurement when one or more of said one or more biometric measurements is within a predetermined region.

### CROSS-REFERENCE TO RELATED APPLICATIONS

This application is a continuation-in-part of U.S. patent application Ser. No. 17/876,744, filed Jul. 29, 2022, published Dec. 1, 2022, which is a continuation-in-part of U.S. patent application Ser. No. 17/334,891, filed May 31, 2021, published Dec. 1, 2022, the disclosures of which are incorporated herein by reference.

### BACKGROUND

Smart watches (or other smart wearable) have the ability to monitor the respiration rate of the user. This measurement is typically used to provide statistics after the fact. There is a need to use these measurements to provide instant feedback to a user to alter their respiration pattern to help with anxiety and relaxation. By taking control of their breathing in real time, the user can impact their well-being all-day long.

### BRIEF SUMMARY

In a first embodiment, a breathing coach system and method is disclosed comprising a biometric device capable of measuring one or more biometric measurements of a user; an application executed on a processor on a wearable device monitoring said one or more biometric measurements coupled to said biometric device and notifying the user to perform a recommended breathing pattern to effect said one or more biometric measurement when one or more of said one or more biometric measurements is within a predetermined region.

In another embodiment, said one or more biometric measurements comprises a heart rate, a blood pressure or body temperature.

In accordance with another aspect, there is provided a monitoring system comprising: at least one wearable breathing sensor; a user device communicatively coupled to the wearable breathing sensor, the user device comprising one or more processors coupled to a user interface and to a memory comprising computer-executable instructions that cause the one or more processors to: receive from each of the at least one wearable breathing sensor a signal related to a breathing event from a user; extract from the signal breathing data comprising a breathing rate and an inhale depth; causing the user interface to display, for each of the at least one wearable breathing sensor, a corresponding plurality of sequential elements; and affecting a visual change in each of the plurality of elements sequentially to indicate a ratio of the inhale depth with respect to a predetermined inhale breath threshold.

In some embodiments, the instructions further cause the one or more processors to: provide a notification to the user via the user device upon the inhale depth not being equal or exceeding the predetermined inhale breath threshold during said breathing event.

In some embodiments, the notification is only provided upon the breathing data indicating the user is not talking.

In some embodiments, the change is reset upon an exhale being detected in said breathing data.

In some embodiments, the instructions cause the one or more processors to define the inhale depth as said predetermined threshold when extracting from a subsequent signal.

In some embodiments, the instructions cause the one or more processors to extract the breathing data using one or more machine learning algorithms.

In some embodiments, the wearable breathing sensor comprises a force sensor coupled to an elastic band.

In some embodiments, the force sensor comprises: a variable impedance coupled to the elastic band; a constant current source; and a voltage amplifier.

In some embodiments, the at least one wearable breathing sensor is configured to monitor diaphragm-based breathing.

In some embodiments, the at least one wearable breathing sensor comprises a first wearable sensor configured to monitor chest-based breathing, and a second wearable sensor configured to monitor diaphragm-based breathing simultaneously, and wherein said inhale depth includes both a chest-based inhale depth, and a diaphragm-based inhale depth; and wherein said inhale breath threshold includes both a chest-based inhale threshold and a diaphragm-based inhale threshold.

In some embodiments, the instructions further cause the one or more processors to: provide a notification to the user via the user device upon the diaphragm-based inhale depth not being equal or exceeding the diaphragm-based inhale breath threshold during said breathing event.

In accordance with another aspect, there is provided a method comprising the steps of: disposing, on a body of a user, at least one wearable breathing sensor; receiving, on a user device communicatively coupled to the at least one wearable breathing sensor, from each of the at least one wearable breathing sensor, a signal related to a breathing event from a user; extracting, by the user device, from the signal breathing data comprising a breathing rate and an inhale depth; causing a user interface of the user device to display, for each of the at least one wearable breathing sensor, a corresponding plurality of sequential elements; and affecting on the user interface a visual change in each of the plurality of elements sequentially to indicate a ratio of the inhale depth with respect to a predetermined inhale breath threshold.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a wearable smart device in accordance with one embodiment.

FIG. 2 illustrates an embodiment of the breathing coach system.

FIG. 3 a schematic diagram of an improved breathing monitoring and coaching system, in accordance with one embodiment.

FIG. 4 is a schematic diagram of a force sensor of the system of FIG. 3, in accordance with one embodiment.

FIG. 5 is a graphical plot illustrating a breathing waveform, in accordance with one embodiment.

FIG. 6 is a graphical plot illustrating a chest-based breathing waveform superimposed over a diaphragm-based breathing waveform, in accordance with one embodiment.

FIG. 7, FIG. 8, and FIG. 9 are examples user interfaces, in accordance with different embodiments.

FIG. 10 illustrates an aspect of the subject matter in accordance with one embodiment.

### DETAILED DESCRIPTION

The details of one or more embodiments of the subject matter of this specification are set forth in the accompanying drawings and the description below. Other features, aspects, and advantages of the subject matter will become apparent from the description, the drawings, and the claims.

Like reference numbers and designations in the various drawings indicate like elements.

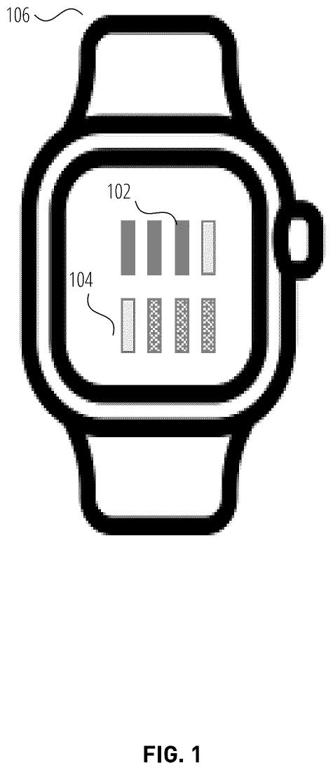

FIG. 1 depicts a wearable smart device 106 capable of measuring respiration rate. An application downloaded on the wearable smart device 106 displays bars reflecting the inhale level 102 and the exhale level 104 of the users as measured and compared to a baseline natural conscious breathing of the user.

In another embodiment, the biometric device is a separate device coupled with the application device via a communication protocol.

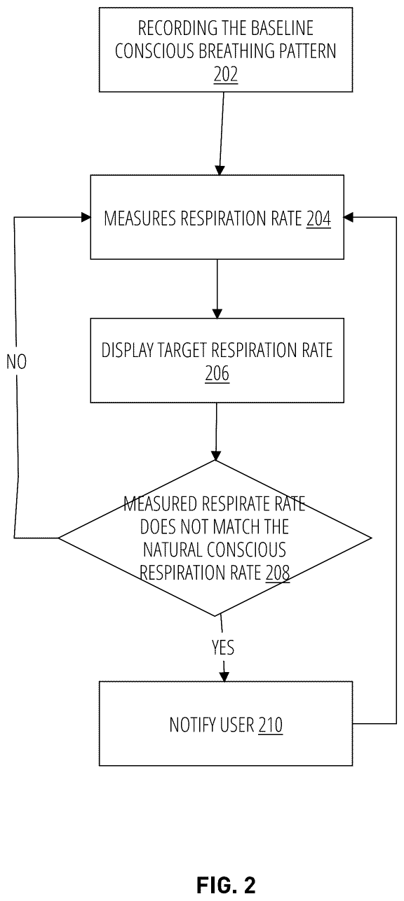

As per FIG. 2, the user initialize the application by recording the baseline conscious breathing pattern 202. The conscious breathing pattern is stored in the wearable smart device 106 as the baseline.

Then the application measures respiration rate 204 and displays target respiration rate 206 by showing the inhale level 102 and exhale level 104 on the screen of the wearable smart device 106. The levels and speed can be shown using bars as shown in FIG. 1. For example, the inhale level 102 is shown with bars lighting up from left to right and the exhale level 104 with the bars going from right to left. If the application is configured to have a pause between the inhale and the exhale, and/or the exhale and the inhale, then another graphic icon can be used as a counter for the pause.

The display could be of any designs that allows to show the two dimensions of the respiration, the rate and the depth.

If the measured respirate rate does not match the baseline 208, the user is notified 210 using a vibration or a sound generated by the wearable smart device 106. For example if the user tries to exhale before the 4th bar of the inhale is reached.

The user can configure the application to make different sounds, types of vibrations and levels thereof.

The application measures respiration rates 204 on an ongoing basis and continuously compares to the baseline to provide continuous feedback. Optionally, the application can be configured via setting to only provide feedback during specific times of day.

As the user goes on about its day and forgets to breath consciously, the watch will constantly remind the user to go back to their baseline natural conscious breathing pattern.

In another embodiment, the application can be configured to implement different types of breathing rhythms including a pause after the inhale and/or before the exhale. Optionally, the application can use different types of vibration for the inhale, pause, exhale. For example the 4,4,4 box breathing technic used navy seals. Any other types of breathing techniques or patterns can be programmed into the application.

Using the application the user does not need to worry about counting and they can focus on the breath and visualize toxic energy leaving their body. A subtle vibration of the wearable smart device 106 on the wrist triggers the user to inhale and hold their breath for 4 seconds, then another vibration will notify the user to exhale. The application acts as a metronome for the breathing of the user causing ongoing real-time relaxation.

In another embodiment, the application on the wearable smart device 106 will cause the user to take deeper breaths than the baseline to increase relaxation. Using this setting the vibration may optionally feel different or a different sound can be played. In this embodiment, the application acts as a metronome for breathing.

In another embodiment, the application records the breathing pattern of the user. Using this data, the application can display the breathing pattern in comparison with previous time segments or in comparison with the baseline conscious breathing pattern to show improvements achieved using the notifications.

In another embodiment, the application provides statistics of the number of time the notifications are sent, these statistics can show the user improvement over time.

In another embodiment, the application uses the heart rate measured by the device 106 to determine whether to execute a predetermined breathing pattern is needed. When the measured heart rate is within a predetermined region (e.g. higher than a first threshold and lower than a second threshold), a breathing pattern is recommended using sound and/or vibration notification on the wearable device. Optionally, multiple predetermined regions can be established, and a different breathing pattern may be used when the heart rate enters a different region. If the user's heart rate lowers to another pre-determined region, the application resets to a normal breathing pattern and stops the notifications.

For example, if the user's heart rate is above 120 bpm and below 150 bpm a first breathing pattern is executed on the wearable device 106. If the heart rate exceeds 150 bpm, a second breathing pattern is executed. When the heart rate falls below 120 bpm, then the breathing notifications stop.

In another embodiment, the application uses the blood pressure measured by the device to calculate which type of predetermined breathing pattern is needed. When the measured blood pressure is within a predetermined region, a breathing pattern is recommended using sound or vibration notification on the wearable device. If the user follows the breathing pattern and the blood pressure lowers to another pre-determined region, the application reset to a normal breathing pattern and stops the notifications.

In another embodiment, before the user takes a blood pressure test the device, the user notifies the application and a predetermined breathing pattern is executed for a predetermined time (e.g. 60 seconds) to lower the blood pressure. For example, the breathing pattern would be a 60 second slower and deep breathing rhythm, vibrating at the end of each inhale and exhale signaling the user to breath the opposite direction, for 60 seconds or until the resting heart rate is below a threshold (e.g. 60 bpm) before the user starts the blood pressure test.

In another embodiment, the application uses the body temperature measured by the device to calculate which type of predetermined breathing pattern is needed. When the measured temperature is within a predetermined region, a breathing pattern is recommended using sound or vibration notification on the wearable device. If the user follows the breathing pattern and the temperature changes to another pre-determined region, the application resets to a normal breathing pattern and stops the notifications.

In another embodiment, the application uses the respiration rate measured by the device to calculate which type of predetermined breathing pattern is needed. When the measured respiration rate is within a predetermined region, a breathing pattern is recommended using sound or vibration notification on the wearable device. If the user follows the breathing pattern and the respiration rate changes to another pre-determined region, the application reset to a normal breathing pattern and stops the notifications.

In another embodiment, the biometric device uses a pulse oximeter, and the respiration rate is inferred from the oxygen saturation.

The recommended breathing pattern may include a combination of longer, deeper, shallower, shorter breath.

Optionally, the application takes into account the activity of the user. The user enters the activity it is doing from a predetermined list (e.g. weight training, running, cycling, working, meditating) using the application interface or voice commands. The application recommends a breathing pattern that is specific to the activity.

As a first example, the application helps a user to lower the heart rate. A user is exercising in a gym, with a heart rate at 170 bpm. However, the user could be preforming the same at 155 beats per minute. The application detects the high heart rate and selects a breathing pattern to help lower the heart rate. The user starts following the recommended breathing pattern (e.g. by taking deeper breaths) and the heart rate lowers.

The breathing application implements the necessary pattern which has a deeper breathing pattern uploaded right into the device 106, the rate, the speed, the depth is all predetermined by the application with the goal to lower the heart rate.

As another example, the application helps lowering high heart rate while relaxing. A user is running the breathing application on their wearable device 106 while relaxing but the resting heart rate is at 75 bmp. The heart rate could be a bit lower given the level of activity they are doing. The application sets a breathing pattern to a specific rate, depth and speed to bring the heart rate down to a set goal of 60 beats per minute. When the set goal of 60 beats per minute is reached the deeper slower breathing pattern reverts to the original predetermined breathing pattern. The original breathing pattern being what the user is uses by default with the application.

As another example, the user sets the application to “exercising” and the heart rate is at 120 beats per minute which is within the predetermined target but the breathing is faster and shallower than the set goals. The application selects and executes a pre-determined breathing pattern that triggers the user to take deeper inhales and exhales with at lower speed between inhale and exhale. The user follows the recommended breathing pattern to get more oxygen and further lowers the hearth rate.

In another embodiment, the application includes voice recognition or a sensor that picks up the voice vibrations thru the wrist. With this feature, the breathing notifications are silences when the user talks, as the user does not breath normally when talking, which would trigger a lot of notifications. Once the user stops talking for a predetermined amount of time, the silent mode is turned off and the user starts to receive breathing notifications again, as described above.

The user of the application sets up several parameters such as age, height, weight, gender, other biometric parameters such as resting heart rate along with predetermined thresholds on the rate and depth that will trigger notifications. Predetermined breathing patterns may be downloaded customized to the user's specific information.

For example, the vibration generated by the wearable device can be done by the face and/or wristband, in the case of a watch.

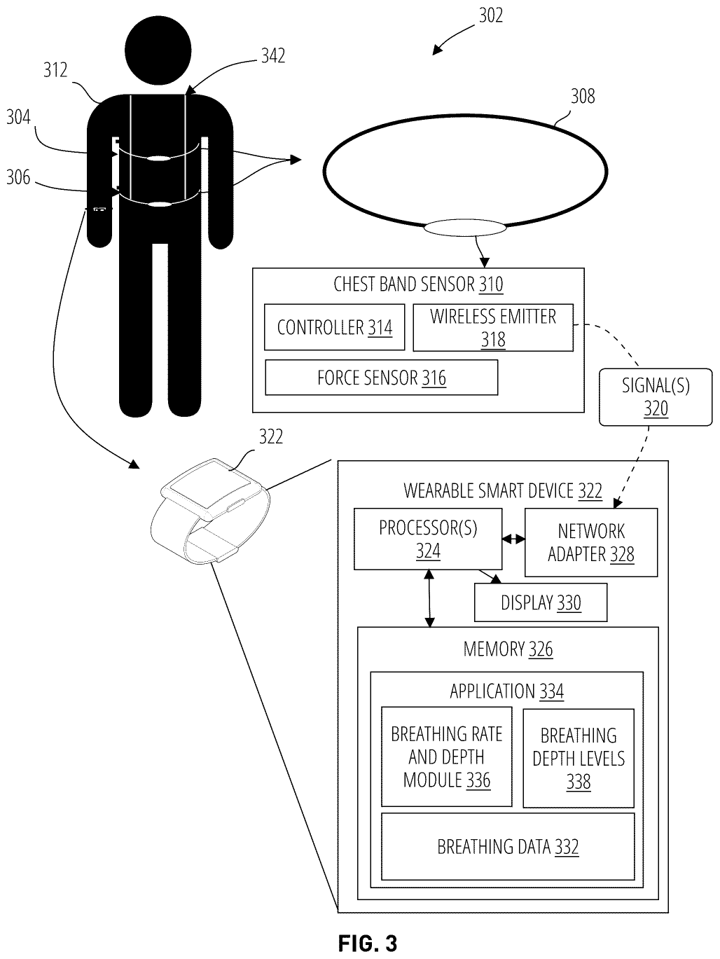

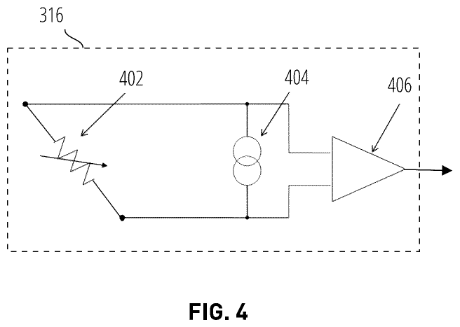

FIG. 3 shows an improved breathing monitoring and coaching system 302, in accordance with one embodiment. In this example, the system 302 comprises a first wearable breathing sensor 304 and optionally but preferably a second wearable breathing sensor 306. If only the first wearable breathing depth sensor 304 is used, it can be sized and configured to be comfortably worn across the user's chest region to monitor intercostal breathing (i.e., chest breathing) only, or across the user's abdominal region to monitor diaphragmatic breathing only. In contrast, if the second wearable breathing sensor 306 is also used, then as illustrated in FIG. 4, the first sensor 304 can be used to monitor chest breathing while the second sensor 306 can be used to monitor diaphragmatic breathing simultaneously. Each breathing sensor similarly comprises an elastic band 308 or similar, and a chest band sensor 310 configured to measure breathing rate and depth based on the variation of the force experienced by the elastic band as a function of time due to chest/abdominal expansions and contractions. The system 302 thus comprises different modes of operations: a normal mode where only chest-based breathing is monitored, an abdominal mode wherein only diaphragm-based breathing is monitored, a third mode (breathing coach mode) that monitors both chest and diaphragm-based breathing. All these modes may further include a supplementary “everyday mode” or a “download mode” as will be discussed below.

The elastic band 302 is configured to provide enough tension to stay in position without being too unconformable to a user 312. In some embodiments, the elastic band 302 may be adjustable to different sizes and/or body types. Additionally, in some embodiments, shoulder straps 342 or similar may also be provided to prevent the bands from slipping. Some embodiments, each band may comprise its own straps, or a single strap may be configured to accommodate two bands.

Each band sensor comprises a controller 314 coupled to a force sensor 316 and a wireless emitter 318. As discussed below, the force sensor 316 is coupled to the elastic band and converts into an electrical signal change in force experienced due to the extension and retraction of the elastic band during breathing. The measured analog signal is digitized by the controller 314 and a wireless signal(s) 320 is sent to a wearable smart device 322 (herein illustrated as a smart watch worn by the user). The signal(s) 320 may be transmitted using any known short range communication technologies (e.g., near-field communication (NFC), BLUETOOTH® branded communications, or the like), or a combination thereof. In some embodiments, additional information or data may be communicated with the signal(s) 320, for example a device ID identifying the wearable sensor (useful when more than one is used).

The wearable smart device 322, such as the illustrated smart watch, typically comprises one or more processors 324 coupled to a memory 326, a network adapter 328 and a digital display 330. Breathing data 332 received from the signal(s) 320 is stored in memory 326 via a software Application 334. Other components may also be present, for example one or more input interfaces, speakers (to generate audible cues), haptic feedback components (to generate vibrations), or the like. The software application 334 comprises instructions that provide further analysis with a breathing rate and depth module 336 and cause the processors to display information/instructions via the digital display 330. While a smart watch is illustrated, it will be appreciated that other personal computing devices may be used as well, for example a personal computer or tablet. The applications 334 may further be configured to memorize if only one or two wearable sensors are being used, and for the latter which wearable sensor is used to monitor the user's chest and which wearable sensor is used to monitor the user's abdomen.

The breathing rate and depth module 336 comprises instructions that when executed by the one or more processors cause the processor to determine from the breathing data 332 a breathing rate and a breathing depth (e.g., amplitude). In the case where the second sensor band 304 is used to monitor diaphragmatic breathing, the module is further configured to determine whether a designated breathing rate and depth is associated with intracoastal breathing or diaphragmatic breathing. A number of breathing depth levels 338 (or thresholds) may also be stored in memory 326. As will be discussed further below, these may be entered by the user, or determined automatically.

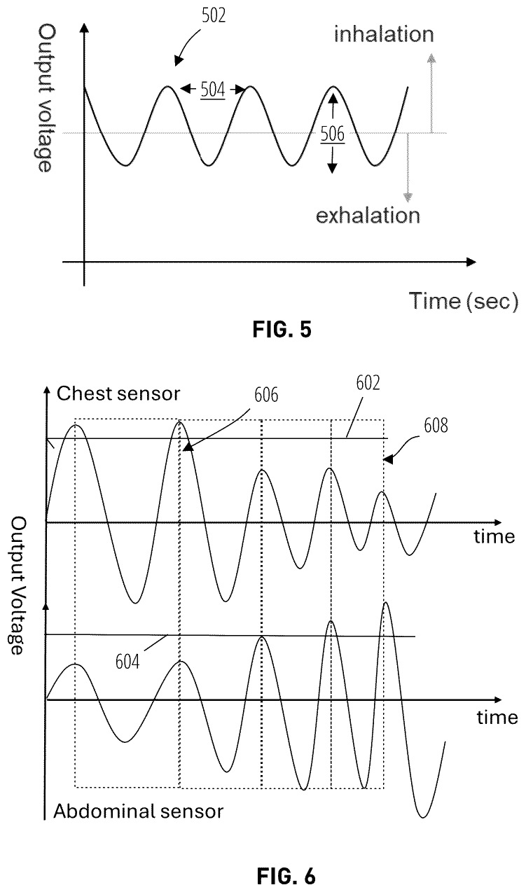

FIG. 4 illustrates a schematic diagram of the force sensor 316, in accordance with one embodiment. The force sensor 316 is used to determine the depth of breathing and the breathing rate and comprises the illustrated analog signal processing circuit. The circuit is shown to include a variable chest band impedance component 402, a constant current source 404, and a voltage amplifier 406. During inhalation, the chest/abdominal volume will expand which creates an increase in force on the force sensor 316 that produces an increase in impedance. During exhalation, the impedance will decrease. FIG. 5 illustrates an exemplary waveform 502 illustrating the output signal from the force sensor 316 (whether it be from chest breathing or diaphragmatic breathing). Breathing rate can be determined by identifying time between two peaks (e.g., 504), and a breathing depth can be determined using the amplitude 506 of the signal during the identified breath or breathing event. In some embodiments, the controller 314 may average a plurality of consecutive measurements to provide a less noisy signal. Other signal processing techniques may be used as well, for example by applying one or more filters to the raw voltage to remove vibrations or other motions not directly related to the user's breathing.

In cases where only the first wearable breathing sensor 304 is used, only one breathing type monitored, and the methods discussed above may be used without limitation. However, if both sensors 304 and 306 are used, then different breathing types may require breathing-specific calibrations, as the signals from the force sensor 316 will differ as a function of the body location. In some embodiments, the system may be configured to calibrate the sensors for each individual user, by providing breathing instructions (breathing in or out, via the chest or diaphragm) and measuring the output of the sensors concurrently. In some embodiments, the applications 334 may be configured to identify automatically whether the signals are related to chest-based breathing or diaphragmatic breathing, and thus to automatically associate a given wearable sensor to a body location of the user. Other embodiments may simply be used directly the

In addition, breathing typically will cause both sensors to measure at least some signal at the same time, and the breathing rate and depth module 336 is configured to determine from both signals if the breathing type can be characterized as intracoastal or diaphragmatic. An example of dual waveforms illustrating this is shown in FIG. 6. Different peak detection algorithms may be considered by the breathing rate and depth module 336 to determine the breathing depth associated with each type of breathing. For example, this may include in some embodiments determining the relative amplitude of the signals received from each sensor, and use for example a user defined threshold value to categorize the measured breathing. In the illustrated example, a inhale level or threshold 602 and 604 is used. This inhale level can be set up manually by the user via the application 334, or can be determined based on a calibration procedure or the like (including the user setting a conscious relaxed breath baseline). In addition, a plurality of sub-thresholds may also be set up. Other examples not illustrated may include using one or more threshold values for each signal. In some embodiments, one or more machine learning algorithms may be used to perform the categorization, including for example gradient boosting which is well suited for feature extraction and classification of time-series data. In some embodiments, this may include using an artificial neural network (ANN), support vector machines (SVMs), random forests or the like trained to perform such determinations using data provided by a plurality of users over time. The neural networks may rely on different architectures, such as a Long Short-Term Memory (LSTM) architecture (well-suited for time-series analysis), or a convolutional neural network (CNN) architecture (performs well for feature extraction, and classification of time-series data). In some embodiments, time series analysis algorithm, for example dynamic time warping (DTW) or longest common subsequence (LCSS) can be used to compare the shape and duration of the inhalation events. In some embodiments, each waveform is calibrated individually and/or independently (such as shown in FIG. 8 or FIG. 9 as discussed below).

In some embodiments, the Application 334 is further configured to present via the display 330 to the user dynamical graphical outputs providing an indication in real-time of the breathing type, breathing rate and breathing depth. This advantageously allows to communicate more efficiently a substantial amount of information in a more concise and efficient way. This provides a notable improvement over similar systems by allowing to transfer a larger amount of information quickly, allowing the system 302 to be used in real-time.

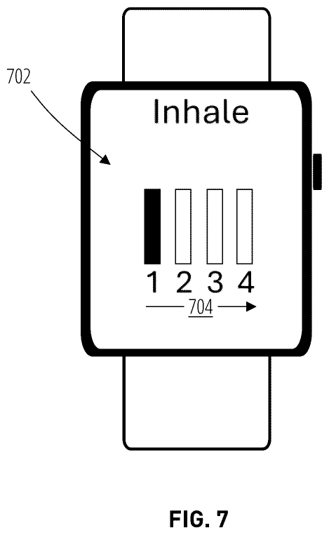

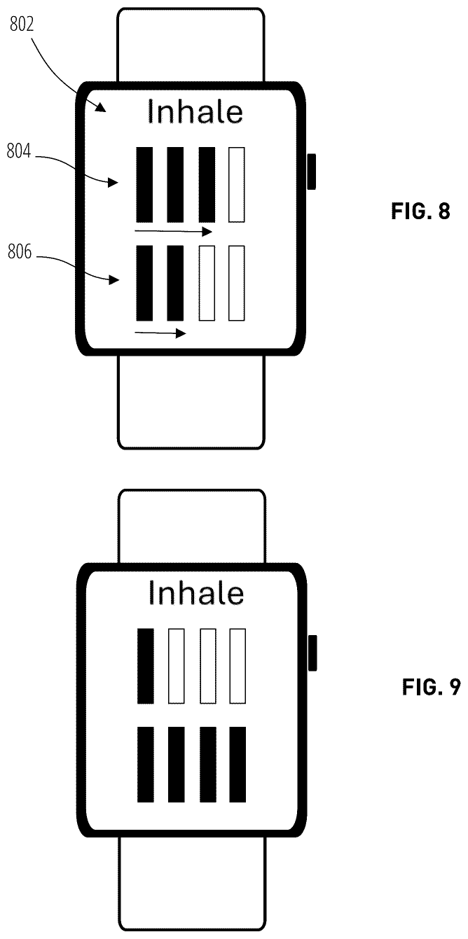

FIG. 7 illustrates an exemplary interface 702 used when only a single wearable sensor is used, either on the chest or on the abdominal region, in accordance with one embodiment. In this example, a single row of four bars are provided to indicate breath level or depth (from left to right). In contrast with the display of FIG. 1 wherein both inhale levels and exhale levels where displayed, the current example only displays inhale levels. It will also be appreciated that a breathing level or depth will be illustrated dynamically by affecting a visual change to the bars in the direction of the arrows 704 (from left to right, although other directions or orientations for the bars may be used as well). Effecting a visual change may include lighting up the bars, but also changing their color, lighting intensity, having the bars appearing and disappearing or other like changes. While this example comprises four vertical bars, it will be appreciated that different numbers of bars may be displayed as well. In some embodiments, the last bar is illuminated/changed upon the user inhale level reaching the pre-determined threshold (for example thresholds 602 or 604 stored in memory 326). The other bars may be configured to display specific percentages of that upper threshold (for example 25% (first bar), 50% (second bar), 75% (third bar)), or each may have its own pre-determined sub-threshold. The graphical representation is reset (no bars are lit up) immediately upon the breathing rate and depth module 336 detecting the user exhaling (e.g., by detecting that a maximum in the signal was reached). Upon the user taking a deeper inhale than the pre-determined threshold, no notification is issued or displayed. However, if the user doesn't reach the threshold in a given breath (for example only has 3 bars or less lighted up), then a notification is issued.

In some embodiments, the system 302 may further include different monitoring and coaching capabilities. One such mode includes an “everyday mode” that monitors inhalation counting to the predetermined threshold as illustrated in FIG. 7 (from bar #1 to bar #4 on the right). As noted above, the threshold (depth of breathing) corresponding to bar #4 may be customizable to accommodate individual breathing needs. In the “everyday” mode, the wearable smart device (via the application 334) only notifies the user when their inhale depth does not reach a certain depth threshold while inhaling. When notified, the user can take a deeper breath on their next inhale, and the notification turns off. This subtle cue encourages to maintain a consistent and optimal breathing depth.

To set a conscious relaxed breath as a baseline or threshold, the user may place the application 334 may be set to a “download mode” and inhales intentionally. This deliberate action allows the device to capture and store in memory 326 the user's relaxed breath pattern (in breathing data 332). This stored baseline breath may serve as a reference point for the device to rack and provide feedback on the user's breathing patterns. Advantageously, the application 334 is configured to synchronize in real-time with the user's real-time breathing depth, providing accurate and precise feedback. This is in contrast with other monitoring applications or devices that only track breathing rates or provide unconnected visual cues.

In some embodiments, setting the conscious relaxed breath may be used with the baseline natural conscious breathing pattern functionality described above wherein both the inhale and exhale levels are displayed to the user. In such examples, the breathing pattern will include both inhale data and exhale data, and the conscious relaxed breath baseline or threshold is determined by the user exhaling (during a breathing event).

FIG. 8 illustrates a similar example of an interface 802 displayed when both chest and abdominal sensors are in use simultaneously. In this example, the interface comprises two rows of vertical bars, each row displaying inhale levels only, but for a different breathing type. The top row 804 is used to communicate (for example) chest breathing information, while the lower row 806 is used to communicate (for example) diaphragm-based breathing information. Thus, when the user inhales from their chest, in this example the top row of bars light up from left to right. When the user inhales from their abdominal section (diaphragm breathing) the bottom row of bars light up from left to right. If the user uses both chest and diaphragm to inhale then both rows of bars light up at the same time. The exemplary bars in FIG. 8 illustrate, for example inhale level 606 in FIG. 6, while FIG. 9 illustrates the inhale level 608 in the same figure (where diaphragmatic breathing is much stronger). In some embodiments, the Application 334 may store in memory 326 the user's pre-set inhale preferences for specific breathing types (chest-based or diaphragm-based). For example, if the user has set a diaphragm-based inhale preference, the Application 334 can be configured to notify them if they are not meeting this target (e.g., breathing too much from their chest). In some embodiments, the GUI can be used to provide coaching instructions to the user, for example to help the user relax or breathe properly. In other uses, it can be used to treat panic attacks or the like.

In configurations where only a single wearable sensor is used, the GUI may then operate similarly to the one described in FIG. 1, however it will be able to monitor inhale/exhale levels and breathing rate for either chest-based breathing or diaphragmatic breathing.

The foregoing descriptions of specific embodiments of the present invention have been presented for purposes of illustration and description. They are not intended to be exhaustive or to limit the invention and method of use to the precise forms disclosed.

Obviously, many modifications and variations are possible in light of the above teaching. The embodiments described were chosen and described in order to best explain the principles of the invention and its practical application, and to thereby enable others skilled in the art to best utilize the invention and various embodiments with various modifications as suited to the particular use contemplated. It is understood that various omissions or substitutions of equivalents are contemplated as circumstance may suggest or render expedient but is intended to cover the application or implementation without departing from the spirit or scope of the claims of the present invention.

What is claimed is:

1. A monitoring system comprising:
at least one wearable breathing sensor;
a user device communicatively coupled to the wearable breathing sensor, the user device comprising one or more processors coupled to a user interface and to a memory comprising computer-executable instructions that cause the one or more processors to:
receive from each of the at least one wearable breathing sensor a signal related to a breathing event from a user;
extract from the signal breathing data comprising a breathing rate and an breathing depth;
causing the user interface to display, for each of the at least one wearable breathing sensor, a corresponding plurality of sequential elements; and
affecting a visual change in each of the plurality of elements sequentially to indicate a ratio of the breathing depth with respect to a predetermined inhale breath threshold.

2. The monitoring system of claim 1, wherein the instructions further cause the one or more processors to:
provide a notification to the user via the user device upon the inhale depth not being equal or exceeding the predetermined breathing breath threshold during said breathing event.

3. The monitoring system of claim 2, wherein said notification is only provided upon the breathing data indicating the user is talking.

4. The monitoring system of claim 2, wherein said change is reset upon an exhale being detected in said breathing data.

5. The monitoring system of claim 1, wherein the instructions cause the one or more processors to define the inhale depth as said predetermined threshold when extracting from a subsequent signal.

6. The monitoring system of claim 1, wherein the instructions cause the one or more processors to extract the breathing data using one or more machine learning algorithms.

7. The monitoring system of claim 1, wherein said wearable breathing sensor comprises a force sensor coupled to an elastic band.

8. The monitoring system of claim 6, wherein the force sensor comprises:
a variable impedance coupled to the elastic band;
a constant current source; and
a voltage amplifier.

9. The monitoring system of claim 1, wherein the at least one wearable breathing sensor is configured to monitor diaphragm-based breathing.

10. The monitoring system of claim 1, wherein the at least one wearable breathing sensor comprises a first wearable sensor configured to monitor chest-based breathing, and a second wearable sensor configured to monitor diaphragm-based breathing simultaneously, and wherein said inhale depth includes both a chest-based inhale depth, and a diaphragm-based inhale depth; and wherein said inhale breath threshold includes both a chest-based inhale threshold and a diaphragm-based inhale threshold.

11. The monitoring system of claim 10, wherein the instructions further cause the one or more processors to:
provide a notification to the user via the user device upon the diaphragm-based inhale depth not being equal or exceeding the diaphragm-based inhale breath threshold during said breathing event.

12. A computer-implemented method, comprising the steps of:
disposing, on a body of a user, at least one wearable breathing sensor;
receiving, on a user device communicatively coupled to the at least one wearable breathing sensor, from each of the at least one wearable breathing sensor, a signal related to a breathing event from a user;
extracting, by the user device, from the signal breathing data comprising a breathing rate and an inhale depth;
causing a user interface of the user device to display, for each of the at least one wearable breathing sensor, a corresponding plurality of sequential elements; and
affecting on the user interface a visual change in each of the plurality of elements sequentially to indicate a ratio of the inhale depth with respect to a predetermined inhale breath threshold.

13. The method of claim 10, further comprising:
providing, by the user device, a notification to the user upon the inhale depth not being equal or exceeding the predetermined inhale breath threshold during said breathing event.

14. The method of claim 10, wherein said change is reset upon an exhale being detected in said breathing data.

15. The method of claim 10, wherein said inhale depth is defined as said predetermined threshold when extracting from a subsequent signal.

16. The method of claim 10, wherein said extracting is done using one or more machine learning algorithms.

17. The method of claim 10, wherein said at least one wearable breathing sensor is disposed on a user's abdomen to monitor diaphragm-based breathing.

18. The method of claim 10, wherein the at least one wearable breathing sensor comprises a first wearable sensor disposed on the user's chest to monitor chest-based breathing, and a second wearable sensor disposed on the user's abdomen to monitor diaphragm-based breathing simultaneously, and wherein said inhale depth includes both a chest-based inhale depth, and a diaphragm-based inhale depth; and wherein said inhale breath threshold includes both a chest-based inhale threshold and a diaphragm-based inhale threshold.

19. The method of claim 18, further comprising:
provide a notification to the user via the user device upon the diaphragm-based inhale depth not being equal or exceeding the diaphragm-based inhale breath threshold during said breathing event.
